(19) United States
(12) Patent Application Publication
(10) Pub. No.: US 2003/0095990 A1
(43) Pub. Date: May 22, 2003

(54) Microemulsion facial washes comprising specific oils

(75) Inventors: Xi Yuan Hua, Peekskill, NY (US); Leonard Van Gorkom, Englewood, NJ (US); Michael Paul Aronson, West Nyack, NY (US); Zhenhe Zhu, Peekskill, NY (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 09/884,388

(22) Filed: Jun. 19, 2001

(51) Int. Cl. A61K 9/00; C09K 3/00

(52) U.S. Cl. 424/400; 516/74

(57) ABSTRACT

The invention relates to microemulsion comprising oils having defined cleansing and spreadability and providing superior cleansing, mildness, and good feel relative to conventional, non-microemulsion products.

### FIELD OF THE INVENTION

The present invention relates to cleansing microemulsion compositions, particularly facial wash compositions, which compositions have superior cleansing power (e.g., as measured by sebum removal and make-up removal) relative to conventional, non-microemulsion products.

### BACKGROUND OF THE INVENTION

Microemulsions have long been recognized as useful in cleaning applications because of their excellent solvent properties, especially for oily soils. They are widely used in industrial and hard surface cleaning. However, several factors have limited their use in applications for cleansing the skin. The first factor is related to mildness or skin compatibility. Because microemulsions have high solvency, for example, microemulsions can strip essential barrier lipids from the skin along with excess sebum and makeup. This leaves the skin over dry and, in the extreme, dull and white. This factor is related both to the type of oil, surfactant, and co-surfactant/co-solvent used.

A second factor that has limited microemulsions use in skin cleansing is the sensory properties of formulation—especially its rinsing characteristics and the residual feel they impart to skin. On one extreme the microemulsion can rinse well from the skin but leave the skin feeling excessively “stripped of oil” and be perceived as drying. On the other extreme, if the oil phase is excessively tenacious to skin, the microemulsion can be difficult to rinse from the skin and leave it feeling excessively oil and sticky—defeating the purpose for using it as a cleanser.

The third factor that has limited use in skin cleansing applications is formulation robustness. It is desirable in modern cleansing systems to incorporate skin benefit ingredients that actually improve the feel and appearance of the skin over time. It is also desirable to include polymers, dispersed solids and other ingredients that modify viscosity, texture or sensory properties. It is well known that microemulsions can be very sensitive to additional ingredients that alter the effective hydrophobic-lipophilic balance (HLB), temperature or balance point of the surfactant/co-solvent systems. For example, the addition of ionic materials can often destabilize a microemulsion or alter its stability as a function of temperature.

Thus, one objective of this invention is to provide microemulsions that remove sebum and make-up very effectively yet do not strip essential barrier lipids or disrupt the protein structure of the stratum corneum, i.e., are highly compatible with the skin.

A second objective of the invention is a microemulsion that removes sebum and make-up effectively, is highly compatible with skin, and at the same time rinses well with water and leaves the skin feeling clean without a sticky or oily residue.

Yet a third objective of this invention is a microemulsion technology that has broad stability and is sufficiently robust to allow the incorporation of a range of functional and sensory benefit agents to be used.

Unexpectedly, applicants have found that, if oils used in the microemulsions are specifically selected by a so-called “spreadability” factor or ratio (i.e., ratio of spreading time of selected oil to spread time of oleyl oleate over a predetermined area), the microemulsions containing said oils not only will provide enhanced cleansing (relative to other facial cleanser products and as measured by ability to remove sebum and make-up) while maintaining skin compatibility, but the microemulsions also will rinse quickly enough to avoid the “oily” feeling associated with higher oil products. In addition, the microemulsions are robust enough to support incorporation of desired functional and sensory agents.

Applicants have further found that optional addition of polyalcohol humectants (to replace at least part of volatile alcohol cosurfactants generally used in microemulsion compositions) helps to modify the microemulsion water structure to provide the benefits noted above while additionally allowing humectant to serve as a skin benefit agent (e.g., in retaining skin moisture).

### BRIEF DESCRIPTION OF THE INVENTION

The present invention relates to facial wash formulations in the form of microemulsion compositions. The microemulsion compositions comprise specifically selected oils defined by their ability to remove sebum and by a so-called “spreadability” factor which spreadability factor affects both cleansing ability and skin feel of the composition. The microemulsions further optionally comprise polyalcohol humectants which substitute for cosurfactants traditionally used in microemulsion formation and which, in addition to providing above noted benefits in a stable microemulsion system, provide a skin benefit agent desirable in such facial wash formulations.

More specifically, the microemulsion compositions of the invention comprise:

(1) 5 to 30%, preferably 8 to 25% by wt. of the microemulsion facial wash compositions of an oil or oils which can dissolve at least 20% triolein and which have a spreadability factor (ratio of spreading time in minutes of selected oil to spreading time in minutes of oleyl oleate) of greater than 0.3 to less than 2.5;

(2) 5 to 40% by wt. of microemulsion of a surfactant selected from the group consisting of anionic surfactants, nonionic surfactants, amphoteric/zwitterionic surfactants, cationic surfactants and mixtures. In preferred microemulsions, there will be at least one nonionic surfactant such as polyoxyethylenated alcohols (e.g., Brij® 30 ex Unichema or Neodol® type surfactants ex Shell) or polysorbate surfactants such as 1,4 sorbitan monoester or triester);

(3) 1 to 15%, preferably 3 to 10% by wt. microemulsion of a water soluble cosurfactant or cosurfactants comprising a C₂ to C₁₀ straight or branched alcohol such as 1-propanol, isoproponal or hexanol;

(4) 0 to 30%, preferably 0.1 to 25%, more preferably 1 to 20% by wt. of microemulsion of water soluble polyalcohol or humectant (for example, C₂-C₆ alkylene glycol, C₂-C₆ alkyl glycerol, glycerol, urea); and

(5) balance of microemulsion, water.

Most, if not all facial wash/skin cleanser products are in the forms of thick liquids, gels, or creams/pastes, and may be foaming or non-foaming. The present invention deals with non-foaming skin/facial cleanser compositions which are in transparent and stable microemulsion form.

### DETAILED DESCRIPTION OF THE INVENTION

The present invention relates to oil in water (O/W), transparent type microemulsions comprising (1) oil phase, (2) water phase, (3) primary surfactant (e.g., anionic, alkoxylated nonionic), and (4) cosurfactant (e.g., short chain alcohol).

The use of microemulsions of the type described have been limited in facial cleansing for reasons noted above.

Unexpectedly, however, applicants have found that, if oils are specifically selected according to defined criteria, they have superior cleansing effect and are not too oily. Specifically, when oils are chosen with specific cleansing ability (defined by ability to dissolve triolein and by spreadability factor as defined), the microemulsions provide oils which are strong cleansers and acceptable to consumers. The oils may also, of course, act as emollients and are not harmful to skin. The compositions are also robust.

Further, applicants have found that, when alcohol surfactants traditionally used in formation of microemulsions are partially replaced by polyalcohols/humectants, the humectants not only provide the same cleansing and good feel benefits, but further provide a way of delivering skin benefit agent (i.e., the humectant itself) in the facial wash composition.

An additional benefit of these facial microemulsions is that the microemulsion changes appearance, with dilution, from a transparent to blue mini-emulsion to milky emulsion. These changes provide visual cues as to efficiency which are highly desirable. Further, the microemulsions provide changes in tactile feel from initially slippery and fluid to later feeling like “bare” skin with a little tautness. Again, such sensory feelings are desirable in that they correspond to consumer expectations for cleansing and make up removal.

In short, the microemulsion composition of the invention are novel, stable, facial wash compositions in which oils are specifically selected (by cleansing efficiency and spreadability) to provide a composition providing both cleansing power and consumer desirable properties (e.g., skin feel). Optional use of humectant provides the same properties while additionally providing the ability to deliver added benefit agent(s), an attribute which is of particular importance in facial/skin wash applications.

Specifically, compositions of the invention comprise:

(1) 5 to 30%, preferably 8 to 25% by wt. of microemulsion composition of an oil or oils which are defined by their ability to dissolve at least 20% triolein (in addition to ability to dissolve oily soils, the oil ideally should provide lubricant and other tactile sensations during wash or drying; the more readily an oil spreads, the less “greasy” the oil is generally perceived to be). Generally oils should have “spreadability” (measured by spreadability factor, S^{R}, defined herein) of 0.3<S^{R}<2.5 although oils with medium spreading (0.5<S^{R}<1) are preferred;

(2) 5 to 40% by wt. microemulsion of primary surfactant (i.e., level of primary surfactant is generally equal or greater than level of cosurfactant or humectant) selected from the group consisting of anionic surfactants, nonionic surfactants, amphoteric/zwitterionic surfactants, cationic surfactants and mixtures thereof. Preferably the composition will comprise at least one nonionic which will be present at levels of 0 to 40% by wt. microemulsion;

(3) 1 to 15%, preferably 3% to 10% by wt. microemulsion of a water soluble cosurfactant(s) comprising C₂-C₁₀ straight or branched alkanol (e.g., propanol, hexanol);

(4) 0 to 30%, preferably 0.1 to 25%, more preferably 1 to 20% by wt. microemulsion composition of a water-soluble polyalcohol or humectant (e.g., C₂-C₆ alkylene glycol, alkyl glycol, glycerol, urea); and

(5) water to balance.

Each of these components is described in further detail below.

Oils

As noted above, the oils used in the microemulsions of the subject invention are defined by at least the following 2 criteria: (1) ability to dissolve oily soils as measured by ability to dissolve 20% or more triolein; and (2) ability to provide lubrication and tactile sensation measured by spreadability factor, S^{R}, of 0.3 to 2.5.

Examples of the types of dissolving, spreadable oils which may be used include:

Isopropanol Myristrate

Cetiol® CC: Dicaprylyl Carbonate

Isopropanol Palmitate

Cetiol® PGL: Hexyl Decanol and Hexyldecyl Laurate

Squalene

Glycerol Tricaprylate

Squalane

Model Sebum

Light Mineral Oil

Olive Oil

Isopropanol Myristrate

Coco Caprylate/Caprate

Dioctyl Cyclohexane

Oleyl Alcohol

Mineral Oil (Low Viscosity)

Octyl Dodecanol

Caprylic/Capric Triglyceride

Oleyl Eructate

Mineral Oil (Viscous)

Almond Oil

Triolein Removal

With regard to the triolein removal, triolein is a natural component of human sebum as seen from the “composition of Model Sebum” noted below:

### Composition of Model Sebum: Total Fatty Acids to Triglycerides=1

| Ingredient | Wt. % |
|---|---|
| Lauric Acid | 11.5 |
| Oleic Acid | 11.5 |
| Isostearic Acid | 5.75 |
| Tricaprin | 11.5 |
| Triolein | 11.5 |
| Glycerol Triisostearate | 5.75 |
| Oleyl Oleate | 10.4 |
| Myristyl Myristate | 10.4 |
| Isostearyl Isostearate | 5.2 |
| Squalene | 12 |
| Cholesterol Oleate | 3 |
| Cholesterol | 1.5 |

In facial cleansing, the dosage of a cleanser on skin is approximately 0.05 ml per cm². Assuming oily skin has 500 μg/cm² sebum, ratio of sebum to cleanser is 1%. Thus, if solely relying on oil solvency to remove sebum, sebum dissolution in a formulation comprising 10% oil should be at least 10%.

According to dissolution test of the invention, the oil chosen must have ability to dissolve 20% or more triolein. Representative oils meeting this test include squalane, squalene, isopropyl myristate (IPM), light mineral oil.

An example of an oil which cannot dissolve even 10% triolein is silicone oil, i.e., not all oils are the same.

Spreadability

As for oil “spreadability factor”, S^{R}, a semi-quantitative assay was developed by applicants to determine the spreading time for the different oils that have the potential to be used in microemulsions. First, an elliptic shape with 12 mm×8 mm diameters was marked on the inner forearm of a panelist. Because of the textural orientation of the skin, the spreading trace is usually in an elliptic shape. Hence, the orientation of the marked area must be carefully decided. According to the test, 4 μL of the tested oil was dosed in the center of the area. The time when the oil arrived at the marked outline was recorded as the spreading time of the oil. The data were obtained on two panelists with duplicate tests for each oil. The average values were taken as the results. The spreading time of oleyl oleate was than used to normalize the measured values. For example, the spreading time for oleyl oleate is 1.3+0.03 minutes for IPM so the relative spreading time for IPM is 0.5/1.3=0.4. Thus, the spreadability factor, S^{R}, is ratio of spread rate of selected oil (measured in minutes) to spread rate of oleyl oleate (also measured in minutes).

Using this test, examples of oil spreading times are noted below:

TABLE I
Oil Spreading Times On Human Inner Forearm

| | *Relative | | **Relative |
|---|---|---|---|
| Chemicals | Spreading Time | Chemicals | Spreading Time |
| Oleyl Oleate (Control) | 1 | Oleyl Oleate | 1 |
| Cetiol ® OE: Di-N-Octyl Ether | 0.2 | Dicaprylyl Ether | 0.3 |
| Isopropanol Myristrate | 0.4 | Isopropanol Myristrate | 0.4 |
| Cetiol ® CC: Dicaprylyl | 0.5 | Coco Caprylate/Caprate | 0.5 |
| Carbonate | | | |
| Isopropanol Palmitate | 0.7 | Dioctyl Cyclohexane | 0.5 |
| Cetiol ® PGL: Hexyl Decanol | 0.7 | Oleyl Alcohol | 0.6 |
| and Hexyldecyl Laurate | | | |
| Squalene | 0.8 | Mineral Oil (Low Viscosity) | 0.6 |
| Glycerol Tricaprylate | 0.9 | Octyl Dodecanol | 0.7 |
| Squalane | 1.0 | Caprylic/Capric Triglyceride | 0.75 |
| Model Sebum | 1.3 | Oleyl Eructate | 1.2 |
| Light Mineral Oil | 1.5 | Mineral Oil (Viscous) | 2.1 |
| Olive Oil | 1.8 | Almond Oil | 2.2 |
| Pentahydrosqualene | 2.5 | Castor Oil | 12.8 |
| Triolein | 3.7 | | |
| Castor Oil | 7.7 | | |

*Data of present work.
**It should be noted that Cognis Care Chemicals also has data for spreading area over fixed time. To double check with applicants' numbers, applicants used data from Cognis and converted into relative spreading time by using oleyl oleate as control. As noted from the data in far right column, the data from Cognis was in approximate agreement.

In general, using the above noted spreadability factor, oil can be categorized by their spread rates as follows:

Type 1: S^{R}<0.5: high spread, provide initial feel but fade quickly;

Type 2: 0.5<S^{R}<1: medium spread, provide best smooth feeling for medium period;

Type 3: S^{R}>1: slow spread, feel greasy with lasting residual effect, high S^{R} is waxy feeling.

According to the subject invention, oils which are used should have spread rate of 3.0<S^{R}<2.5, preferably 0.5<S^{R}<1. Oils which are 0.3 and below are too “fast” and, although they may provide a desired feeling, the feeling will fade quickly. On the other hand, oils which are 2.5 and above are too “slow” and will feel greasy and sticky.

The oil will comprise 5% to 30% by wt., preferably 8 to 25% by wt. of microemulsion.

Surfactants

The primary surfactant (comprising 5-40%, preferably 10 to 30% by wt. of the microemulsion) is selected from the group consisting of anionic surfactants, nonionic surfactants, amphoteric/zwitterionic surfactants, cationic surfactants and mixtures thereof.

The anionic detergent active which may be used may be aliphatic sulfonates, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS); or aromatic sulfonates such as alkyl benzene sulfonate.

The anionic may also be an alkyl sulfate (e.g., C₁₂-C₁₈ alkyl sulfate) or alkyl ether sulfate (including alkyl glyceryl ether sulfates). Among the alkyl ether sulfates are those having the formula:

RO(CH₂CH₂O)ₙSO₃M

wherein R is an alkyl or alkenyl having 8 to 18 carbons, preferably 12 to 18 carbons, n has an average value of greater than 1.0, preferably greater than 3; and M is a solubilizing cation such as sodium, potassium, ammonium or substituted ammonium. Ammonium and sodium lauryl ether sulfates are preferred.

The anionic may also be alkyl sulfosuccinates (including mono- and dialkyl, e.g., C₆-C₂₂ sulfosuccinates); alkyl and acyl taurates, alkyl and acyl sarcosinates, sulfoacetates, C₈-C₂₂ alkyl phosphates and phosphates, alkyl phosphate esters and alkoxyl alkyl phosphate esters, acyl lactates, C₈-C₂₂ monoalkyl succinates and maleates, sulphoacetates, alkyl glucosides and acyl isethionates.

Sulfosuccinates may be monoalkyl sulfosuccinates having the formula:

R⁴O₂CCH₂CH(SO₃M)CO₂M; and

amide-MEA sulfosuccinates of the formula;

R⁴CONHCH₂CH₂O₂CCH₂CH(SO₃M)CO₂M

wherein R⁴ ranges from C₈-C₂₂ alkyl and M is a solubilizing cation.

Sarcosinates are generally indicated by the formula:

R′CON(CH₃)CH₂CO₂M,

wherein R¹ ranges from C₈-C₂₀ alkyl and M is a solubilizing cation.

Taurates are generally identified by formula:

R²CONR³CH₂CH₂SO₃M

wherein R² ranges from C₈-C₂₀ alkyl, R³ ranges from C₁-C₄ alkyl and M is a solubilizing cation.

Particularly preferred are the C₈-C₁₈ acyl isethionates. These esters are prepared by reaction between alkali metal isethionate with mixed aliphatic fatty acids having from 6 to 18 carbon atoms and an iodine value of less than 20. At least 75% of the mixed fatty acids have from 12 to 18 carbon atoms and up to 25% have from 6 to 10 carbon atoms.

Acyl isethionates, when present, will generally range from about 10% to about 70% by weight of the total composition. Preferably, this component is present from about 30% to about 60%.

The acyl isethionate may be an alkoxylated isethionate such as is described in Ilardi et al., U.S. Pat. No. 5,393,466, hereby incorporated by reference.

Amphoteric surfactants which may be used in this invention include at least one acid group. This may be a carboxylic or a sulphonic acid group. They include quaternary nitrogen and therefore are quaternary amido acids. They should generally include an alkyl or alkenyl group of 7 to 18 carbon atoms. They will usually comply with an overall structural formula:

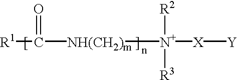

where R¹ is alkyl or alkenyl of 7 to 18 carbon atoms;

R² and R³ are each independently alkyl, hydroxyalkyl or carboxyalkyl of 1 to 3 carbon atoms;

m is 2 to 4;

n is 0 to 1;

X is alkylene of 1 to 3 carbon atoms optionally substituted with hydroxyl, and

Y is —CO₂— or —SO₃—

Suitable amphoteric surfactants within the above general formula include simple betaines of formula:

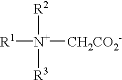

and amido betaines of formula:

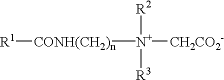

where n is 2 or 3.

In both formulae R¹, R² and R³ are as defined previously. R¹ may in particular be a mixture of C₁₂ and C₁₄ alkyl groups derived from coconut so that at least half, preferably at least three quarters of the groups R¹ have 10 to 14 carbon atoms. R² and R³ are preferably methyl.

A further possibility is that the amphoteric surfactant is a sulphobetaine of formula:

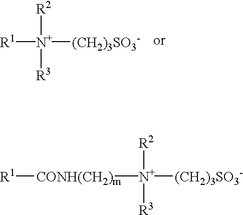

where m is 2 or 3, or variants of these in which —(CH₂)₃ SO₃⁻ is replaced by

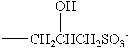

In these formulae R¹, R² and R³ are as discussed previously.

The nonionic which may be used as the second component of the invention include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic surfactant compounds include long chain tertiary amine oxides, long chain tertiary phosphine oxides and dialkyl sulphoxides.

The nonionic may also be a sugar amide, such as a polysaccharide amide. Specifically, the surfactant may be one of the lactobionamides described in U.S. Pat. No. 5,389,279 to Au et al. which is hereby incorporated by reference or it may be one of the sugar amides described in U.S. Pat. No. 5,009,814 to Kelkenberg, hereby incorporated into the subject application by reference.

Examples of cationic surfactants are the quaternary ammonium compounds such as alkyldimethylammonium halogenides.

Other surfactants which may be used are described in U.S. Pat. No. 3,723,325 to Parran Jr. and “Surface Active Agents and Detergents” (Vol. I & II) by Schwartz, Perry & Berch, both of which are also incorporated into the subject application by reference.

In preferred embodiments, the microemulsions will comprise at least one nonionic surfactant. Nonionic surfactants are generally preferred because of their low irritation tendency to human skin. Preferred nonionics include polyoxyalkylenated alcohols such as those having the formula R—(OCH₂CH₂)ₙ where n is 2 to 30 and R is straight or branched chain alkyl. Specific examples include Brij® 30 from Unichema or Neodol® 20-7 from Shell. Other examples include sorbitan and sorbitol esters and their derivative (e.g., Tween® 80 from Unichema or Span®80 from Unichema)

Cosurfactants

The compositions of the invention will also comprise 1 to 15%, preferably 2 to 10%, more preferably 3 to 7% by wt. microemulsion of a cosurfactant which is generally a volatile alcohol. Specifically, this cosurfactant is a C₂ to C₁₀, preferably C₂ to C₆ branched or straight chain alcohol. Examples include propanol, e.g., 1-proponal, and hexanol.

Polyalcohol/Humectant

In addition to the co-surfactant, microemulsion compositions of the invention also include an optional humectant to at least partially replace some of the co-surfactant.

The humectant/polyalcohol provide mildness as moisturizer. Further it can replace part of cosurfactant and act as cosurfactant.

In addition, the humectant, itself being a benefit agent, plays the dual role of allowing stable incorporation of oil into microemulsion and of benefit agent delivery.

The water soluble polyalcohol or humectant may include C₂-C₆ alkylene glycol (preferably propylene glycol) or alkyl glycols; glycerol and urea.

Other examples include any of the humectants noted below:

Alcaligenes polysaccharides; algae extract; aloe barbadensis leaf extract; bacillus/rice bran extract/soybean extract ferment filtrate; betaine; black strap powder; diglycereth-7 malate; diglycerin; diglycol guanidine succinate; erythritol; fructose; glucose; glucoronolactone; glycereth-7 glycolate; glycerin; glyceryl dimaltodextrin; glycol; hesperetin laurate; 1,2,6-hexanetriol; honey; hydrogenated honey; hydrogenated starch hydrolysate; hydrolyzed wheat protein/PEG-20 acetate copolymer; hydroxypropyltrimonium hyaluronate; inositol; lactic acid; lacitol; maltitol; maltose; mannitol; mannose; methoxy PEG-7; methoxy PEG-10; methoxy PEG-16; methoxy PEG-25; methoxy PEG-40; methoxy PEG-100; PEG 4; PEG-6; PEG-7; PEG-8; PEG-9; PEG-10; PEG-12; PEG-14; PEG-16; PEG-18; PEG-20; PEG-32; PEG-40; PEG-45; PEG-55; PEG-60; PEG-75; PEG-90; PEG-75; PEG-90; PEG-100; PEG-135; PEG-150; PEG-180; PEG-200; PEG-220; PEG-240; PEG-800; PEG-15 butanediol; PEG-3-methyl ether; PEG-4 methyl ether; PEG-5 pentaerythrityl ether; polyglyceryl sorbitol; potassium dextrin octenylsuccinate; potassium PCA; PPG-6 sorbeth-245; PPG-6 sorbeth-500; propylene glycol; rosa canina seed extract; sea water; sodium acetylated hyaluronate; sodium dextrin octenylsuccinate; sodium glucuronate; sodium PCA; sorbeth-6; sorbeth-20; sorbeth-30; sorbeth-40; sorbitol; sorbityl silanediol; sucrose; TEA dextrin octenylsuccinate; trehalose; triglycereth-7 citrate; trioxaundecanedioic acid; tripropylene glycol; urea; urea-d-glucuronic acid; xylitol; xylose.

Humectants may comprise 0 to 30%, preferably 0.1 to 20%, more preferably 1-20% by wt. microemulsion.

Water

Finally, to the extent the microemulsions are oil-in-water emulsions, the continuous phase of the emulsion is water.

Sensory Additives

In preferred embodiments, the microemulsions may additionally comprise 0.5 to 10% “sensory” agents. Among the sensory agents which may be added include: (this is a non-exhaustive list and is not intended to be limited):

(1) silicone polymers and oils (e.g., dimethicone);

(2) plant glycosides such as saponins (e.g., tea-seed saponins);

(3) menthols;

(4) α-hydroxy acids (e.g., lactic acid) and β-hydroxy acids (e.g., salicylic acid);

(5) menthol esters;

(6) perfumes;

(7) scrubbing materials (e.g., beads, nutshell, etc.);

(8) thickeners.

Among thickeners which may be used are cross-linked acrylic acid/methacrylate copolymers such as Carbopol series (ex. B. F. Goodrich). Another thickener is PEG iso-pentaerithrityl tetrastearate (PEG-PET).

Other optional ingredients include preservatives, dyes, particles for scrubbing.

Properties

The microemulsion composition of the invention may be further defined on the basis of various properties as noted below.

(1) the compositions are transparent, remain stable between 5° and 45° C.;

(2) the microemulsion can undergo freeze-thaw process;

(3) the microemulsion can dissolve at least 1% sebum, preferably at least 2% sebum, while staying in microemulsion form. That is, as noted earlier, 10% oil, if dissolving sebum of an amount of 10% (i.e., through mechanism of sebum dissolution in oil) should dissolve 1% sebum (assuming cleanser is used at 0.05 ml cm² and there is 500 μg/cm² sebum in oily skin)

Regarding stability (item (1) above), microemulsions were placed in 45° C. oven and a 5° C. oven respectively for four months. Batches at 45° C. were stable and remained transparent Batches at 5° C. remained liquid in the lower portion but had solid floats on top. However, they melted to transparent liquid when sitting at room temperature. Also, several freeze-thaw tests were executed an, in all cases, microemulsion returned when thawed.

Particle size of the microemulsions of the invention is typically 10 to 150 nanometers (nm).

For example, particle size of ME #1 and ME #12 were determined by freeze fracture Cryo-Electron Microscopy. The size range is within 20-80 nm for ME #1, and 20-140 nm for ME #12. They fall well within the size range (10-150 nm) of typical microemulsions.

Interfacial tension of microemulsion and their dilution (1:1) with model sebum was determined by plate method, provided by, for example, Kruss Tensiometer. The value is typically less than 1 dyne/cm, significantly lower than the saturated solutions of many facial cleansers.

Surface tension was measured by drop volume method. Here, 0.2 ml syringe (e.g., from Gilmont® Industries) with a 0.5% reading accuracy was filled with liquid to be tested and set vertically. A drop of liquid was allowed to hang at the glass tip, which had a radius of 0.1995 cm, for at least 5 minutes before falling. Then volume of the drop was recorded. Drop volume is a function of surface tension of liquid as shown.

γ=vρg/2πrf

where γ is surface tension of the tested liquid, v is volume of the liquid drip in ml, ρ is density of liquid, g is gravity constant, r is radius of syringe tip and f is a correction factor. For Interfacial tension, density is replaced by Δρ, which is the density difference between water and the oil.

Typically, surface tension is in a range of 22 to 26 dyne/cm. This is in the range of saturated solutions of most commercial facial cleansers.

The microemulsions all form emulsions spontaneously when diluted (mixed) with water. The diluted solution has Tyndall effect, i.e., reflection, blue light, indicating oil droplets in the emulsion are in the range of colloidal dispersions. Surface tension generally increases slightly with dilution.

The Table below lists surface tensions and appearance for various dilutions.

TABLE 6
Surface Tensions of Micro-Emulsions After Dilution (23° C.)

| | | |
|---|---|---|
| | Surface Tension | |
| Dilution Fold | dyn/cm | Appearance of Solution |

ME #1 (pH 4.5)

| | | |
|---|---|---|
| 0 | 24.3 | Clear & transparent |
| 1 | 25.4 | Bluish, |
| 3 | 26.6 | |
| 6 | 26.3 | |
| 9 | 27.0 | |

ME #2

| | | |
|---|---|---|
| 0 | 21.9 | Clear & transparent |
| 1 | 24.2 | Bluish, slightly opaque |
| 3 | 26.0 | |
| 6 | 26.2 | |
| 9 | 26.2 | |

ME #11

| | | |
|---|---|---|
| 0 | 26.4 | Clear & transparent |
| 1 | 26.8 | Translucent, bluish |
| 3 | 27.4 | |
| 6 | 27.5 | |
| 9 | 27.3 | |

The ARES Rheometer manufactured by Rheometric Scientific™ was used for measuring viscosities in strain controlled mode. Fluid behavior of the microemulsions is different from conventional skin cleansers. They are Newtonian liquids, so the viscosity is constant with varying shear rate or time. Table 7 shows the viscosity at 10 s⁻¹ shear rate for the tested samples. As noted, the viscosities of the two microemulsions are much lower than for the competitors' liquid cleansers. However, low viscosity may ease penetration into crevices. Make-up removal efficacy may be directly related to product viscosity, i.e., an increased viscosity of a product caused a reduction in make-up removal. Hence, low viscosity of the microemulsions may benefit make-up removal. Pond's Clear Solutions® Deep Pore Foaming cleanser has an even lower viscosity than the microemulsions. It is packaged in a bottle with aeration upon dispensing, providing a thick and creamy foam. Microemulsions can be used as a cleansing milk or impregnated onto a non-woven.

To simulate the effect of sebum dissolution in washing process on the viscosity of the microemulsion, 2% model sebum was dissolved in microemulsion #1. A clear fluid was obtained, and its viscosity was slightly increased, as shown in the Table 7. From the results, one can expect that the good properties of the microemulsion, i.e., solvency and low viscosity, will be maintained during the washing process.

TABLE 7
Viscosities of Microemulsions at 20-23° C.

| | | Viscosity at |
|---|---|---|
| | | Shear Rate |
| No. | Samples | 10 s⁻¹ mPa-s |
| 1 | ME #1 | 62 |
| 2 | ME #7 | 95 |
| 3 | 2% Model Sebum in ME #1 | 78.5 |
| 4 | Pond's Clear Solutions ® Deep Pore Foaming | 15 |
| | Cleanser | |
| 5 | Sea Breeze ® Foaming Face Wash | 2,230 |
| 6 | Neutrogena Deep Clean ® Facial Cleanser | 11,451 |
| 7 | Neutrogena ® Oil-free Acne Wash | 7,157 |
| 8 | Water | 1 |

In general, combination of low interfacial tension, spontaneous emulsification and solvency of oils in microemulsion are believed to be helpful for enhanced cleansing.

Methodology

Although mixing order of ingredients is not critical for microemulsion formulation, generally it is preferred to prepare separately an aqueous stock solution and an oil stock solution followed by mixture.

For example, the aqueous solution (Phase A) may comprise thickener (e.g., PEG/PET) dissolved in water. pH is adjusted to about 9.5 to 10 using, for example, NaOH solution. Preservative (e.g., Glydant) and colorant (e.g., Acid Blue) may be added resulting in pH about 7.

The oil stock solution (Phase B) may comprise oil (e.g., dimethicone) mixed with co-surfactant and humectant (e.g., 1-propanol and propylene glycol) followed by nonionic (e.g., Brij 30®) and other oil (e.g., squalane) and gentle stirring.

Phase A and Phase B would be mixed by gentle stirring and pH of final product is about 4 to 7.

Except in the operating and comparative examples, or where otherwise explicitly indicated, all numbers in this description indicating amounts or ratios of materials or conditions or reaction, physical properties of materials and/or use are to be understood as modified by the word “about”.

Where used in the specification, the term “comprising” is intended to include the presence of stated features, integers, steps, components, but not to preclude the presence or addition of one or more features, integers, steps, components or groups thereof.

The following examples are intended to further illustrate the invention and are not intended to limit the invention in any way.

Unless indicated otherwise, all percentages are intended to be percentages by weight.

### EXAMPLES

### Example 1

### Facial Wash Microemulsions with Improved Cleaning of Crevices and Make-up Removal

This example illustrates a facial wash formulation, which is a microemulsion system of oil in water (O/W). It provides substantially improved skin cleaning and make-up removal compared to conventional facial cleansers. The oil is squalane (or squalene), the surfactant is Brij 30, the co-surfactants are propylene glycol (humectant) and 1-propanol. The solutions are transparent. The typical compositions and the range are listed in Table I.

TABLE I
Compositions of Microemulsion Base

| Ingredient | Conc. Range (%, w/w) | Typical Conc. (%, w/w) |
|---|---|---|
| Squalane | 5-30 | 10 |
| Brij 30 | 15-40 | 25 |
| 1-Propanol | 3-7 | 3.5 |
| Propylene Glycol | 3-10 | 6.5 |
| Water | To 100 | To 100 |

### Example 2

### Facial Wash Microemulsion with Improved Cleaning of Crevices and Lubricant Feeling After Wash

This example illustrates a facial wash formulation, which is a microemulsion system of oil in water (O/W) with 0.5% Amodimethicone SF1708 (Aminopropylenepoly-dimethylsiloxane). It provides lubricant and delicate feeling after washing, in addition to effective cleansing. The base formulation of the microemulsion is within the range of Table I.

### Example 3

### Facial Wash Microemulsion with Tea Seed Saponins

This example illustrates a facial wash formulation, which is a microemulsion system of oil in water (O/W) with 5.5% Tea Seed Saponins (about 70% activity). This formulation provides three benefits in addition to effective cleansing: (1) delicate tea-smell (2) anti-microbial effect; and (3) anti-inflammatory. The base formulation of the microemulsion is within the range of Table I.

### Example 4

### Facial Wash Microemulsion with Menthol or Lactic Acid Menthyl Ester

This example illustrates a facial wash formulation, which is a microemulsion system of oil in water (O/W) with 0.5-2% menthol or lactic acid menthyl ester. The formulation provides cooling feeling on skin after wash, which is the stimulus of cleanness and freshness. The base formulation of the microemulsion is within the range of Table I.

### Example 5

### Facial Wash Microemulsion with Perfume

This example illustrates a facial wash formulation, which is a microemulsion system of oil in water (O/W) with 1-2% perfumes, such as Iceberg®. The products may be packed in the bottles with spray heads. The delicate smell and transparent appearance are unique over the conventional products in facial wash. The base formulation of the microemulsion is within the range of Table I.

### Example 6

### Facial Wash Microemulsion with Improved Crevices Clean and Acne Treatment/Prevention

This example illustrates a facial wash formulation, which a microemulsion system of oil in water (O/W) with 0.5% salicylic acid. Squalane and salicylic acid have been used in acne treatment or prevention. The formulation is a “Acne Wash”, providing both effective cleansing and acne treatment/prevention. The base formulation of the microemulsion is within the range of Table I.

### Example 7

### Microemulsion Plus Dispersion in Facial Wash

This example illustrates a facial wash formulation, containing 0.5% Dimethicone/Vinyl SFE839; 25% Brij 30; 10% squalane; 4.4% 1-propanol; 3.6% propylene and 56.5% deionized water. The product is a transparent solution with star-like dispersed particles.

The product cleanses and removes make-up in one step, and leaves skin feeling clear after wash.

An acrylic acid polymer, Carbopol® ETD 2050,can be used to adjust the viscosity of the above formulation. A preservative, Glydant Plus, and a dye, Acid Blue #9 (Erioglaucine), can also be added. The solution is transparent. The pH value of the added polymer solution is critical to maintain a stable microemulsion.

PEG-150 Pentaerthrityl tetrastearate can be used to adjust the viscosity of the above formulation. A preservative, Glydant Plus, and a dye, Acid Blue #9 (Erioglaucine), can also be added. The solution is transparent.

The table below summarizes the seven above-noted microemulsions and another (#8) with lactic acid.

TABLE 3
Microemulsions With Beneficial Additives

| | | | Propylene | Additive | Cloud | |
|---|---|---|---|---|---|---|
| | Additive | 1-Propanol | Glycol % | Range % | Temperature* | pH |
| ME #1 | No | 3.5 | 6.5 | NA | 52 | 4.5-5.1 |
| ME #2 | Amodimethicone | 3.7 | 6.6 | 0.5-1 | 53 | 5.0 |
| | SF 1708 | | | | | |
| ME #3 | Tea Seed | 3.6 | 6.5 | 5.5 | >60 | 5.1 |
| | Saponins | | | | | |
| ME #4 | Menthol | 4.5 | 3.6 | 0.5 | 50 | |
| | | 4.5 | 3.6 | 1 | 49 | 5.1 |
| | | 5.6 | 3.8 | 2 | 40 | 5.2 |
| | Lactic Acid | 4.8 | 3.7 | 1 | 50 | 4.9 |
| | Menthyl Ester | 4.4 | 3.8 | 2 | 45 | 5.1 |
| ME #5 | Iceberg ® | 4.5 | 3.6 | 1 | 48 | 5.5 |
| ME #6 | Salicylic Acid | 3.5 | 6.5 | 0.5-1 | >60 | 3.3-3.6 |
| ME #7 | Dimethicone/ | 4.4 | 3.6-3.8 | 0.5 | 50 | 5.2 |
| | Vinyl SFE 839 | | | | | |
| ME #8 | L (+) Lactic Acid | 3.5 | 6.6 | 2.5 | 45 | 5.6 with |
| | | | | | | 1% NaOH |
| | | | | | | (28%) |

*Cloud Temperature of microemulsion is highest temperature for transparent liquid. To test, microemulsion in glass vial was made and placed in oven where temperature was raised gradually from 25° to 60° C. When solution turns cloudy or there was phase separation, this was read as cloud temperature.

### Example 9

### Procedure to Make Microemulsion Cleansers

Although the order of adding the ingredients is not critical for microemulsion formation, it is recommended to prepare an aqueous stock solution and an oil stock solution. Then, mix the two solutions together. For example, the procedure to prepare the microemulsion with Carbopol in Example 7 is as follows:

(1) Aqueous stock solution (Phase A)—Dissolve 0.3 g of Carbopol® ETD 2050 into 100 g water, adjusted to pH=9.5 to 10 by NaOH. Add 0.3-0.5% of Glydant Plus and 5-10 ppm Acid Blue 9;

(2) Disperse the dimethicone into 1-propanol and propylene glycol, then add Brij 30 and squalane, mixing by gentle stirring—Phase B;

(3) Add Phase A into Phase B, mixing by gentle stirring. The pH of the final product is 4.5.

The procedure to prepare the microemulsion with PEG-150 Pentaerthrityl tetrastearate in Example 7 is as follows:

(1) Aqueous stock solution (Phase A)—Dissolve 0.5 g of PEG-150 Pentaerthrityl tetrastearate into 100 g water, adjusted to pH=9.5 to 10 by NaOH. Add 0.3-0.5% Glydant Plus and 5-10 ppm Acid Blue 9. The pH value of the resulted solution is 6.9;

(2) Disperse the dimethicone into 1-propanol and propylene glycol, then add Brij 30 and squalane, mixing by gentle stirring—Phase B.

(3) Add Phase A into Phase B, mixing by gentle stirring. The pH of the final product is 5.2.

Formulations with low actives and similar performances are always desirable. Table 4 lists three compositions comprising of 15-20% Brij 30 and 5-10% squalane.

TABLE 4
Low Active Formulations, in wt. %

| | | | | | | Cloudy |
|---|---|---|---|---|---|---|
| | | | | | | Tem- |
| | Squa- | | 1- | Propylene | | pera- |
| | lane | Brij ®30 | Propanol | Glycol | Water | ture |
| ME #9 | 10 | 20 | 3.8 | 7.0 | 59.2 | 44 |
| ME #10 | 8 | 20 | 3.9 | 7.2 | 60.9 | 49 |
| ME #11 | 5 | 15 | 4.3 | 8.0 | 67.7 | 43 |

### Example 10

It is normally extremely difficult to produce a single phase ME with surfactant (e.g., nonionic surfactant Tween 80), oils (e.g., IPM), humectant (e.g., glycerol) and water. Through trial and error, however, high glycerol containing microemulsion (ME #12) was successfully prepared. It comprises 17.7% Tween® 80, 2.3% Span 80, 10% isopropanol myristate, 7.1% hexyl alcohol, 3.7% 1-Propanol, 23.7% glycerol and 35.5% water.

### Example 11

In order to show enhanced cleansing, applicants conducted experiment as follows:

Evidence Showing Significantly Better Performance

(1)In Vitro Sebum Removal

High Resolution Image Analysis was used in the tests. The sebum used in the tests was artificial. The applied dosage was 500 μg/cm². After applying a cleansing agent on the soiled spot (2 cm diameter) of a pig-skin, the agent was rubbed gently 30 times in about 30 seconds by use of a finger with Latex glove. For the commercial products, the glove was wet by water before rubbing. No pre-wetting step was used for the microemulsions. The cleansing effectiveness of a product was expressed by the sebum removal % after washing by the product and rinsing by a tap water. Table III below lists the wash effectiveness on porcine skin for the microemulsions and competitors' products.

TABLE III
Cleansing Effectiveness

| | | |
|---|---|---|
| | | Sebum |
| No. | Product | Removal % |

Microemulsions

| | | |
|---|---|---|
| 1 | ME #1 pH 5.8 | 93 |
| 2 | ME #1 pH 4.6 | 92 |
| 3 | ME #2 (Amodimethicone SF 1708) | 89 |
| 4 | ME #3 (Tea Seed Saponins) pH 5.1 | 91 |
| 5 | ME #4 (Menthol) | 90 |
| 6 | ME #5 (Iceberg) | 90 |
| 7 | ME #6 (Salicylic Acid) pH 3.6 | 91 |
| 8 | ME #7 (0.5% Dimethicone/Vinyl SFE | 89 |
| | 839 & Thickener | |
| 9 | ME #7 (0.5% Dimethicone/Vinyl SFE 839) | 91 |
| 10 | ME #8 (2.5% Lactic Acid) | 91 |
| 11 | ME #12 (High Glycerine Containing | 85 |
| | Microemulsion) | |
| 12 | ME #11 (Lower Actives) | 90 |
| 13 | ME #1, followed by Pond's Toner | 94 |

Competitor's Facial Cleansers

| | | |
|---|---|---|
| 1 | Biore ® Cool | 78 |
| 2 | Biore Extra Mild | 74 |
| 3 | Biore Soft Scrub | 84 |
| 4 | Biore Man's Scrub | 84 |
| 5 | Page One Facial Cleanser Double Scrub & | 87 |
| | Cool for Men | |

Unilever Products

| | | |
|---|---|---|
| 1 | Pond's ® Shine Control Facial Gel | 79 |
| 2 | Hazeline ® Deep Cleansing Gel | 71 |
| 3 | Pond's Fair & Lovely Facial Foam | 87 |
| 4 | Pond's Milk + Toner (Wiped off by tissue) | 86 |

In general, sebum removal may be defined into 3 categories as noted below.

| | Removal % |
|---|---|
| High removal | 85% and up |
| Medium | 75-84% |
| Low | Below 75% |

As seen, the percentage of sebum removed (measure of cleansing effectiveness) was consistently higher than that removed by noted products when a microemulsion form was used. Specifically, for all ME, removal was 85% to 93% while, for competitor products, removal was in range of 74-87% and in range 71-87% for applicants' own products (i.e., non-microemulsion products).

### Example 12

The measurement of the Transepidermal Water Loss is one of the most important parameters for evaluating the efficiency of the water barrier of skin. The base formulation (Example #1) was tested on six panelists. They sat in the instrument room at 25° C. and 40% humidity for 20 minutes before the measurements. The TEWL tests were conducted at three points on an arm for each panelist. The Water Loss was determined before washing and after washing by the microemulsion. Table V lists the change of the water loss before and after washing. A negative/positive value indicates increased/decreased barrier for water loss after wash, compared with the natural skin status. The average change of the Water Loss for the six panelists is −0.098 g/hm², which indicates that the skin remains in the original status after washed. Hence, the tested microemulsion is mild, having no damage to epidermal protein and lipid organization. Since the surfactant in the microemulsion is a nonionic, it has very low Zein value, indicating low tendency of skin irritation.

TABLE V
Transepidermal Water Loss

| Panelist Name | Change in Water Loss, g/hm² | | | |
|---|---|---|---|---|
| Position on Arm | Upper | Middle | Low | Average |
| 1 | −1.62 | 0.53 | 1.44 | 0.12 |
| 2 | 0.31 | 2.02 | 2.16 | 1.50 |
| 3 | −0.13 | 0.42 | 0.45 | 0.25 |
| 4 | −0.19 | −2.54 | 0.03 | −0.90 |
| 5 | −0.07 | 0.29 | −2.3 | −0.69 |
| 6 | −3.71 | 0.81 | 0.59 | −0.77 |
| Average | | | | −0.08 |

The data shows the average change in TEWL for the six panelists was −0.08 g/hm², which indicates that the skin remained in the original status after it was washed. Hence, the tested microemulsion does not damage the epidermal proteins and lipid organization of the stratum corneum.

### Example 13

The microemulsion in Example 7 (PEG-150 Pentaerthrityl tetrastearate as the thickener) was used in the make-up removal tests. The benchmarks were Pond's Faire & Lovely Cleansing Milk (CM)/Face Toner and Pond's Cleansing and Make-up Remover Towelettes (Wipe). Revlon Colorstay Liquid Lip (Chianti-24) and Revlon Colorstay Lashcolor Waterproof Black were used as the standard soils. The microemulsion showed a significantly higher power over the references in removal of smudge-proof lipstick and mascara.

1. A microemulsion composition comprising:
(a) 5 to 30% by wt. microemulsion of an oil or oils which dissolve at least 20% triolein and which have spreadability factor S^{R} of greater than 0.3 to less than 2.5;
(b) 5 to 40% by wt. microemulsion of a surfactant selected from the group consisting of anionic, nonionic amphoteric/zwitterionic, cationic and mixtures thereof;
(c) 1 to 15% microemulsion of water-soluble cosurfactant comprising C₂-C₁₀ straight or branched chain alcohol;
(d) 0 to 30% water soluble polyalcohol or humectant; and
(e) balance water.

2. A composition according to claim 1, wherein said oil has spreadability factor of >0.5 to <1.0.

3. A composition according to claim 1, wherein composition comprises nonionic surfactant.

4. A composition according to claim 3, wherein nonionic is polyoxyalkylenated alcohol.

5. A composition according to claim 1, comprising 3 to 10% by wt. C₂-C₁₀ alcohol.

6. A composition according to claim 1, comprising 0.1 to 25% by wt. water soluble polyalcohol or humectant.

7. A composition according to claim 6, wherein humectant is C2-C6 alkylene glycol, C2-C6 alkyl glycerol or urea.

8. A composition according to claim 1, additionally comprising 0.5 to 10% sensory agent.
